(19) United States
(12) Patent Application Publication
(10) Pub. No.: US 2018/0263628 A1
(43) Pub. Date: Sep. 20, 2018

(54) SURGICAL CLIPS AND CONTROLLED DEPLOYMENT THEREOF

(71) Applicant: CLIPTIP MEDICAL LTD., Yokneam Illit (IL)

(72) Inventor: Yehuda Bachar, Givaat Shmuel (IL)

(73) Assignee: CLIPTIP MEDICAL LTD., Yokneam Illit (IL)

(21) Appl. No.: 15/981,910

(22) Filed: May 17, 2018

Related U.S. Application Data
(63) Continuation-in-part of application No. PCT/IB2016/056988, filed on Nov. 20, 2016.
(60) Provisional application No. 62/257,841, filed on Nov. 20, 2015.

(51) Int. Cl. A61B 17/122; A61B 17/00

(52) U.S. Cl. CPC A61B 17/122; A61B 17/1285; A61B 17/00234

(57) ABSTRACT

Surgical clips, clip appliers, and applications thereof, configured for controlled deployment of the clips in a subject's body. The clips and clip appliers are shiftable through several configurations, including a stowed configuration in which clip arms are in proximity and unlocked, a deployed configuration in which clip arms are distant and become lockable, and a locked configuration in which the clip arms are in proximity and locked. In exemplary embodiments, a first clip arm adjoins the other with a flexible extension, optionally curved with an inflection point, and the other clip arm adjoins the first clip arm with a rigid extension. A clip interconnecting mechanism for connecting to another clip may be configured for autonomous release when ejecting from the clip applier.

### RELATED APPLICATION

This application is a continuation-in-part of PCT Patent Application No. PCT/IB2016/056988, which claims priority of U.S. Provisional Patent Application No. 62/257,841, filed Nov. 20, 2015, entitled “Modified Hemostatic Clips”, the contents of which are incorporated herein by reference in their entirety.

### FIELD OF THE INVENTION

The present invention, in some embodiments thereof, relates to surgical clips (including hemostatic clips), clip appliers, and applications thereof, suitable for ligating bodily organs or tissues in a subject.

### BACKGROUND

During many surgical procedures blood vessels or other tubular structures are ligated and cut. Prior art includes various teachings about devices and techniques applicable for ligating bodily organs or tissues, such as blood vessels, among other possible bodily organs or tissues in a subject. Hemostatic clips are well known and commonly used in such teachings. Prior art also includes various teachings about using medical devices and procedures for applying surgical clips. For example, PCT Intl. Appl. Pub. No. WO 2015/040621 A1, of same applicant/assignee as the present invention, discloses a laparoscopic clip applier which includes multiple clips housed in a rigid sleeve, where the arms of the clips are oriented lengthwise in the sleeve, and a deployment mechanism for deploying the clips from a distal end of the sleeve via a perforation made by a needle provided with the distal end of the sleeve.

In spite of extensive teachings in the field and art of the invention, and in view of significant limitations and potential problems associated with such teachings, there is an on-going need for developing and implementing new or/and improved surgical clips, clip appliers, and applications thereof, that are affective in overcoming such limitations or problems in medical procedures of, or involving, ligating bodily organs or tissues in a subject.

### SUMMARY OF THE INVENTION

The present invention, in some embodiments thereof, relates to surgical clips, clip appliers, and applications thereof, suitable for ligating bodily organs or tissues in a subject. In exemplary embodiments, the surgical clips may be endoscopic or/and hemostatic clips (‘hemoclips’) formed with asymmetrical structure for providing secure and efficient bodily organ or tissue ligation. Exemplary embodiments of the present invention also relate to methods of deploying surgical clips.

According to an aspect of some embodiments of the present invention, there is provided a surgical clip comprising: a first clip arm pivotally connected to a second clip arm at a proximal connection region, the first and second clip arms have locking units including mating locking members configured to engage with each other, first of the locking members faces toward a distal end of the surgical clip and second of the locking members faces towards a proximal end of the surgical clip, each of the locking units further includes a contacting surface oppositely facing a respective one of the mating locking members; the surgical clip has a stowed configuration in which the first and second clip arms are in proximity and the locking units are elastically stressed in a deformation such that the contacting surfaces contact each other and the mating locking members are non-engaged with each other; the surgical clip is autonomously shiftable from the stowed configuration to a deployed configuration in which the first and second clip arms are distant from each other and the locking units are unstressed, wherein the first and second clip arms are forcible from the deployed configuration into a locked configuration in which the locking units are absent of the deformation and the mating locking members are engaged with each other.

According to some embodiments, the second clip arm adjoins the proximal connection region with a flexible extension, and the first clip arm adjoins the proximal connection region with a rigid extension.

According to some embodiments, the flexible extension is curved with an inflection point.

According to some embodiments, the surgical clip further comprising an interconnecting mechanism for connecting to another surgical clip. According to some embodiments, the interconnecting mechanism comprises a distal tilting element extending from a distal portion of the surgical clip juxtaposing to one of the clip arms, and a proximal tilting element extending from a proximal portion of the surgical clip juxtaposing to the one of the clip arms, wherein one of the distal and proximal tilting elements tilts towards another of the clip arms, and another of the distal and proximal tilting elements tilts away from the another clip arm. According to some embodiments, the proximal surgical clip portion extends from the first clip arm proximally to the proximal connection region. According to some embodiments, in the stowed configuration, the distal tilting element is parallel to the proximal tilting element. According to some embodiments, the distal tilting element or/and the proximal tilting element is elastically bendable. According to some embodiments, the interconnecting mechanism comprises a concave interconnecting member extending from one of the distal and proximal surgical clip portions, and a convex interconnecting member extending from another of the distal and proximal surgical clip portions.

According to some embodiments, in the stowed configuration, the contacting surfaces are smooth and configured for sliding with each other.

According to an aspect of some embodiments of the present invention, there is provided a surgical clipping system, comprising an introducer with an introducer lumen and an introducer distal tip; and at least one of the surgical clip; wherein in the stowed position, the flexible extension is disposed in the introducer lumen and wherein the clip is deployable out of the lumen to the deployment configuration in which the introducer distal tip contacts the flexible extension adjacent the inflection point thereby allowing the second clip arm pivoting away from the first clip arm about the inflection point.

According to some embodiments, in the deployment configuration, the first clip arm is According to some embodiments, in the deployment configuration, the introducer is movable with respect to the the surgical clip to move the second clip arm towards the first clip arm and engage the mating locking members with each other.

According to an aspect of some embodiments of the present invention, there is provided a surgical clipping system, comprising: a first and a second of the surgical clip, interconnected with each other, wherein the distal tilting element contacts the proximal tilting element and the convex interconnecting member is received in the concave interconnecting member.

According to an aspect of some embodiments of the present invention, there is provided a surgical clip, comprising a first clip arm pivotally connected to a second clip arm at a proximal connection region, wherein the second clip arm adjoins the proximal connection region with a flexible extension, and the first clip arm adjoins the proximal connection region with a rigid extension.

According to some embodiments, the flexible extension is curved with an inflection point.

According to some embodiments, when the flexible extension is kept pressed against the first clip arm, from proximally to the inflection point, the first and second clip arms are forcible into a locked configuration in which the first and second clip arms are locked with each other.

According to some embodiments, when the flexible extension is kept pressed against the first clip arm, across the inflection point, the first and second clip arms are forcible into a stowed configuration in which the first and second clip arms are unlockable with each other.

According to an aspect of some embodiments of the present invention, there is provided a surgical clip, comprising a first clip arm pivotally connected to a second clip arm at a proximal connection region, and an interconnecting mechanism for connecting to another the surgical clip; the interconnecting mechanism comprises a distal tilting element extending from a distal portion of the surgical clip juxtaposing to one of the clip arms, and a proximal tilting element extending from a proximal portion of the surgical clip juxtaposing to the one of the clip arms, wherein one of the distal and proximal tilting elements tilts towards another of the clip arms, and another of the distal and proximal tilting elements tilts away from the another clip arm.

According to some embodiments, the second clip arm adjoins the proximal connection region with a flexible extension, and the first clip arm adjoins the proximal connection region with a rigid extension.

According to some embodiments, the proximal surgical clip portion extends from the first clip arm proximally to the proximal connection region.

According to some embodiments, the distal tilting element or/and the proximal tilting element is elastically bendable.

According to some embodiments, the interconnecting mechanism comprises a concave interconnecting member extending from one of the distal and proximal surgical clip portions, and a convex interconnecting member extending from another of the distal and proximal surgical clip portions.

According to some embodiments, the surgical clip being integrally structured and manufactured from a single piece of material.

According to an aspect of some embodiments of the present invention, there is provided a clip applier, comprising: an introducer including an introducer distal tip and an introducer lumen extending along a longitudinal axis, the introducer is configured for housing at least one clip including a distal-most clip comprising a first clip arm adjoining a proximal connection region with a rigid extension, and a second clip arm adjoining the proximal connection region with a flexible extension; and a pusher configured for advancing the at least one clip to protrude the distal-most clip at a selected distance from the introducer distal tip; wherein the introducer is configured such that the first clip arm stiffly aligns with the longitudinal axis, and the second clip arm is allowed to pivot away from the first clip arm, when the pusher protrudes the distal-most clip at the selected distance.

According to some embodiments, the flexible extension is curved with an inflection point, wherein the introducer distal tip contacts the flexible extension adjacent the inflection point when the pusher protrudes the distal-most clip at the selected distance.

According to an aspect of some embodiments of the present invention, there is provided a method for delivering or/and deploying a surgical clip in a body of a subject, the method comprising:
providing a clip applier, comprising an introducer with an introducer distal tip and an introducer lumen extending along a longitudinal axis, and configured for housing at least one surgical clip including a distal-most surgical clip comprising a first clip arm adjoining a proximal connection region with a rigid extension, and a second clip arm adjoining the proximal connection region with a flexible extension;
advancing the distal-most surgical clip to protrude at a selected distance from the introducer distal tip, while aligning the first clip arm along the longitudinal axis and allowing the second clip arm to pivot away from the first clip arm; and
guiding the clip applier in the subject's body using the aligned first clip arm.

According to some embodiments, the method further comprising dissecting a bodily organ or tissue in the subject's body using the first clip arm.

According to some embodiments, the guiding includes observing distal portion extending from the first clip arm in relation to a target bodily organ or tissue in the subject's body.

According to some embodiments, the method further comprising:
surrounding the bodily organ or tissue with the distal-most surgical clip;
gradually closing the surgical clip over the bodily organ or tissue by forcibly decreasing distance between the first and second clip arms; and
locking together the first and second clip arms.

According to some embodiments, the flexible extension is curved with an inflection point, wherein the advancing includes positioning the introducer distal tip so as to contact the flexible extension adjacent the inflection point.

According to some embodiments, the flexible extension is curved with an inflection point, wherein the closing and locking includes positioning the introducer distal tip distally to the inflection point.

According to some embodiments, the method further comprising ejecting the distal-most surgical clip from the clip applier.

According to some embodiments, the distal-most surgical clip includes an interconnecting mechanism extending from the first clip arm proximally to the proximal connection region, comprising a proximal tilting element juxtaposing to one of the clip arms and a deflected interconnecting member juxtaposing to another of the clip arms and opposing the proximal tilting element.

According to some embodiments, the aligning includes gripping the deflected interconnecting member and elastically bending the proximal tilting element towards the deflected interconnecting member, wherein the ejecting includes releasing the proximal tilting element.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.
In the drawings:

FIG. 1 is a schematic side view showing an exemplary surgical clip, in accordance with some embodiments of the invention;

FIGS. 2A-2C are schematic side views of an exemplary surgical clip, in different deployment configurations thereof, in accordance with some embodiments of the invention;

FIG. 3A is an illustration showing an isometric view of an exemplary surgical clipping system that includes an exemplary clip applier and at least one surgical clip, in accordance with some embodiments of the invention;

FIG. 3B is a block diagram representation of the exemplary surgical clipping system, illustrating an exemplary functional arrangement of components in the exemplary clip applier carrying a plurality of surgical clips, in accordance with some embodiments of the invention;

FIGS. 4A-4D are schematic side cut views of a plurality of sequentially chained hemostatic clips carried in a clip applier, representing exemplary steps in a method for deploying a surgical clip, in accordance with some embodiments of the invention.

### DETAILED DESCRIPTION

The present invention, in some embodiments thereof, relates to surgical clips, clip appliers, and applications thereof, suitable for ligating bodily organs or tissues in a subject. In exemplary embodiments, the surgical clips may be endoscopic or/and hemostatic clips (‘hemoclips’) formed with asymmetrical structure for providing secure and efficient bodily organ or tissue ligation. Exemplary embodiments of the present invention also relate to methods of deploying surgical clips.

While known hemostatic clips are often associated with drawbacks including premature or accidental release of clips from the clip applier during surgery, and/or difficulty in visualizing and controlling accurate positioning of clips over a bodily tissue or organ, the hemostatic clips of the present invention aim to overcome those drawbacks. The hemostatic clips of the invention are structures such that one clip arm is rigidly and stably fixed and aligned with the longitudinal axis of the clip applier when housed within the clip applier and during the process of ligating a bodily tissue or organ and the second clip arm is movable relative to the first clip arm. This particular and unique structure provides security against accidental dislodgment and affords efficient bodily tissue or organ ligation. The hemostatic clips and clip appliers of the invention, are further capable of guiding in an efficient manner the clip applier to a target bodily organ or tissue and/or dissecting the target bodily organ or tissue.

The surgical (e.g., hemostatic) clips of the invention include a first clip arm pivotally connected to a second clip arm at a proximal connection region (e.g., shared proximal end), and may further include at least one of:
an interconnecting mechanism suitable for chaining lengthwise, one after the other a plurality of clips, and
a locking mechanism for locking together the clip arms over a bodily tissue or organ.

The interconnecting mechanism aims to provide increased safety and efficiency of the deployment and clipping process by stably aligning one of the clip arm members with the clip applier's longitudinal axis while ligating a bodily tissue or organ. As such, it may achieve precise bodily tissue or organ ligation and prevent accidental projection of the clips from the clip applier. The locking mechanism aims to provide secure and reliable clip lockdown over a bodily tissue or organ. The locking mechanism may be small in size, minimizing the dimensions of the clip applier of the invention. Furthermore, exemplary surgical clips are characterized in their ability to conform to various tissue thicknesses.

In some embodiments, the first and second clip arms have locking units including mating locking members configured to engage with each other, first of the locking members faces toward a distal end of the surgical clip and second of the locking members faces towards a proximal end of the surgical clip, each of the locking units further includes a contacting surface oppositely facing a respective one of the mating locking members.

In some embodiments, the surgical clip has a stowed configuration in which the first and second clip arms are in proximity and the locking units are elastically stressed in a deformation such that the contacting surfaces contact each other and the mating locking members are non-engaged with each other.

In some embodiments, the surgical clip is autonomously shiftable from the stowed configuration to a deployed configuration in which the first and second clip arms are distant from each other and the locking units are unstressed. When in the deployed configuration, the surgical clip is configured such that wherein the first and second clip arms are compressible into a locked configuration in which the locking units are absent of the deformation and the mating locking members are engaged with each other. In some such embodiments, the surgical clip (i.e., arms thereof) cannot be locked, as in the locked configuration, by not first shifting from the stowed configuration to the deployed configuration.

In some embodiments, the second clip arm adjoins the proximal connection region with a flexible extension, and the first clip arm adjoins the proximal connection region with a rigid extension. The flexible extension is optionally curved with an inflection point.

In some embodiments, the surgical clip includes an interconnecting mechanism for connecting to another surgical clip. The interconnecting mechanism includes a distal tilting element extending from a distal portion of the surgical clip, juxtaposing to one of the clip arms (e.g., the second clip arm), and a proximal tilting element extending from a proximal portion of the surgical clip juxtaposing to the one of the clip arms (e.g., the second clip arm). One of the distal and proximal tilting elements tilts towards another of the clip arms (e.g., the first clip arm), and another of the distal and proximal tilting elements tilts away from the another clip arm (e.g., the first clip arm). Optionally, the proximal surgical clip portion extends from the first clip arm proximally to the proximal connection region. Optionally, when the surgical clip is in the stowed configuration, the distal tilting element is parallel to the proximal tilting element. Optionally, the distal tilting element or/and the proximal tilting element is elastically bendable.

In some embodiments, the interconnecting mechanism includes a proximal deflecting (e.g., concave) interconnecting member extending from the proximal surgical clip portion. The interconnecting mechanism may also include a distal deflecting (e.g., convex) interconnecting member extending from the distal surgical clip portion, in order to grip (e.g., by receiving or/and engaging) the proximal deflecting interconnecting member of a distally adjacent surgical clip.

According to some embodiments, the surgical clip may have a longitudinal length within a range of between about 2 mm and about 50 mm.

According to some embodiments, the diameter of the surgical clip may be less than about 7 mm, less than about 5 mm, less than about 4 mm, less than about 3 mm, less than about 2 mm, or less than about 1 mm. Each possibility represents another exemplary embodiment of the invention.

According to some embodiments, the surgical clip may be integrally structured and manufactured from a single piece of material. According to other embodiments, the surgical clip may be structured and/or manufactured from a set of components being connected one to another, each may be made from a different material.

According to some embodiments, the surgical clip is integrally structured and manufactured from a single piece of material and presenting various elasticities of portions within the clip, which elasticities depend on the dimensions (thickness and/or length) of each of those portions. According to some embodiments, suitable elastic materials may include, but are not limited to, elastic type polymers, elastic metals, elastic metal alloys (e.g. nickel-titanium, stainless steel, cobalt chrome), or a combination thereof.

According to some embodiments, the surgical clip includes a first clip arm and a second clip arm pivotally linked at a shared proximal end.

According to some embodiments, at least one of the first and second clip arms includes an outer rigid clip arm member connected to an inner resilient elastic clip arm member.

According to some embodiments, inner resilient elastic clip arm member is having a tissue contacting surface.

According to some embodiments, the surgical clips of the invention present three clip configurations, selected from: a stowed (e.g., pre-deployed, optionally stressed) configuration, a deployed (e.g., less/non-stressed, fully opened) configuration, and a locking configuration.

The surgical clips of the invention, when shifting from a non-stressed fully opened configuration to a (stressed) locked configuration, apply compressive forces on the bodily organ or tissue being ligated.

It is to be understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. The following exemplary embodiments may be described in the context of exemplary embolization procedures for ease of description and understanding. However, the invention is not limited to the specifically described devices and methods, and may be adapted to various clinical applications without departing from the overall scope of the invention.
According to some embodiments, when ligating a bodily organ or tissue, one clip arm is aligned with a longitudinal axis of the clip applier and the other clip arm is movable relative to the aligned clip arm.

According to some embodiments, in the deployed configuration the first and second clip arms are distanced from each other by a predetermined distance.

According to some embodiments, the predetermined distance is the maximal distance or height displayed between the clip arms. According to some embodiments, the predetermined distance is between about 2 millimeters and about 20 millimeters.

According to some embodiments, in the stressed locked clip configuration the distal ends of the clip arms are in proximity or contact each other.

According to some embodiments, the locking mechanism of the surgical clip is configured for applying resistive force to at least one of the clip arm distal ends, thereby locking together the clip arm distal ends during the locked configuration, so as to prevent self-opening of the surgical clip.

According to some embodiments, in response to ligating a bodily tissue or organ, the clip arms conform to a compressed and clamped shape of the ligated bodily organ or tissue.

According to some embodiments, conformity includes that at least one of the inner resilient elastic clip arm members apply compression and clamping forces on the bodily organ or tissue while allowing at least a portion thereof to expand towards a respective outer rigid clip arm member.

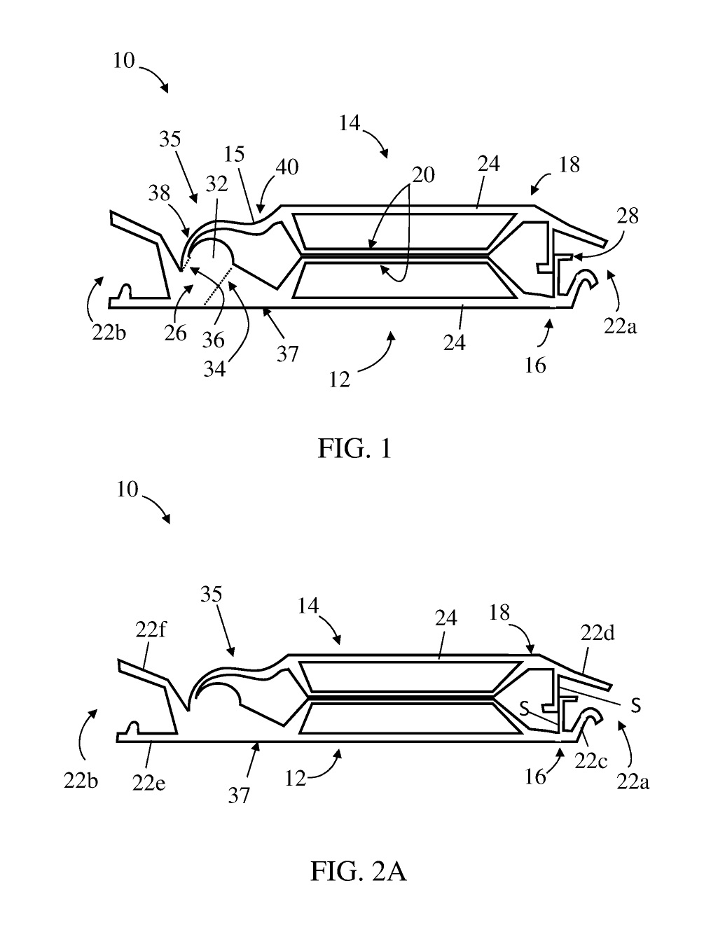

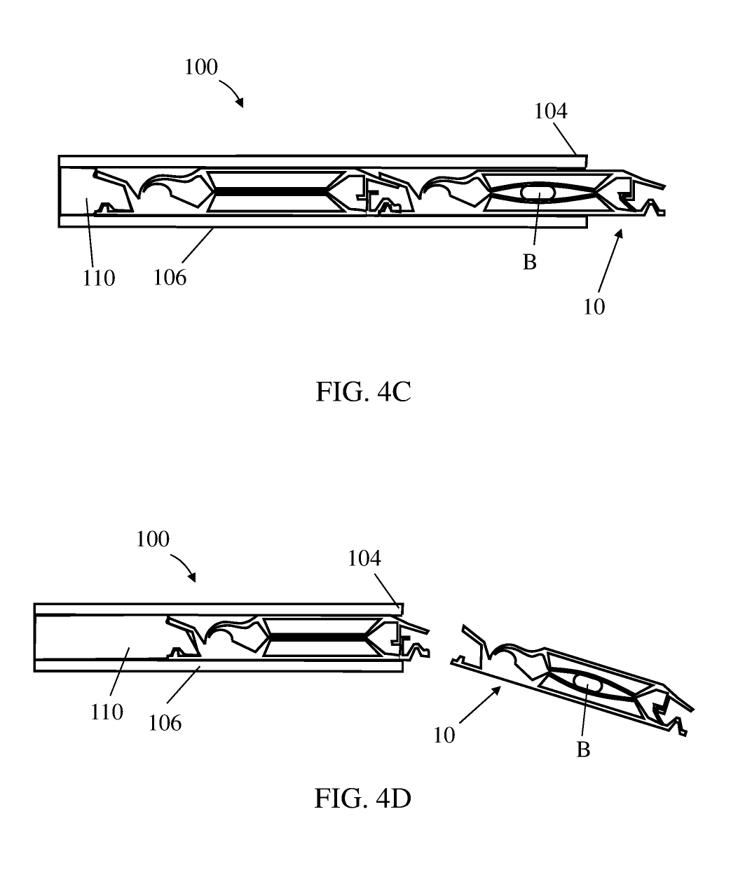

Referring now to the drawings, FIG. 1 is a schematic side view showing a hemostatic (surgical) clip according to some embodiments of the invention. Hemostatic clip 10 includes a first clip arm 12 and a second clip arm 14. First clip arm 12 includes a first clip arm distal end 16 and second clip arm 14 includes a second clip arm distal end 18. Hemostatic clip 10 further includes locking mechanism 28 for locking together first and second clip arms 12 and 14 following a bodily organ or tissue ligation. Hemostatic clip 10 is shown in a stowed configuration, where it is confined to lumen boundaries of an introducer (e.g., in a form of sleeve or tube) of a clip applier (e.g., introducer 106 of clip applier 100, shown in FIG. 4).

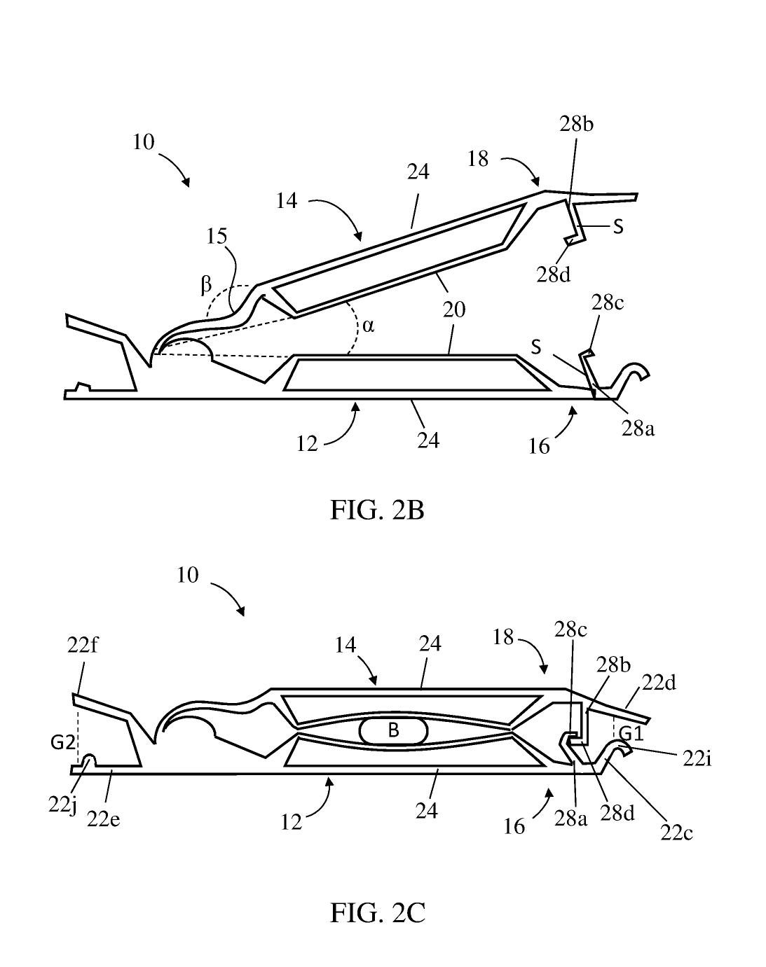

Hemostatic clip 10 may further present a configuration selected from: (i) a stowed configuration, wherein first clip arm distal end 12 is distanced from second clip arm distal end 14 by a predetermined distance (described in more details with respect to FIG. 2B below), and (ii) a locking configuration, wherein first clip arm distal end 16 is in proximity with or contacts second clip arm distal end 18 (shown in FIG. 2C).

Locking mechanism 28 is capable of applying resistive force to at least one of first clip arm distal end 16 and second clip arm distal end 18, upon locking first clip arm distal end 16 with second clip arm distal end 18. Locking mechanism 28 further configured to prevent self-opening of hemostatic clip 10 following a bodily tissue or organ ligation. Locking mechanism and its components will be described in more details with respect to FIG. 2B.

Second clip arm 14 opposes and is pivotally connected to first clip arm 12 at a proximal connection region 26. Proximal connection region 26 is configured for allowing motion (e.g., pivotal) of second clip arm 14 relative (e.g., away or towards) to first clip arm 12.

Optionally, at least of one of first and second clip arms 12 and 14 is elastic along a length extending between proximal connection region 26 and the corresponding first and second clip arm distal ends 16 and 18.

Hemostatic clip 10 further includes an interconnecting mechanism 22 for connecting lengthwise, one after the other a plurality of hemostatic clips 10. Interconnecting mechanism 22 includes a head coupler 22a and a tail coupler 22b. Tail coupler 22b extends proximally from first clip arm 12. Optionally, tail coupler 22b may be directly and/or stiffly connected to a proximal portion of first clip arm 12. Tail coupler 22b may be connected to or emerge directly from first clip arm 12. Head coupler 22a of one hemostatic clip 10 is configured to connect tail coupler 22b of another hemostatic clip 10 in a releasable manner to thereby form a sequential lengthwise chain of a plurality of hemostatic clips 10 within a hemostatic clip applier.

Proximal connection region 26 may include at least one of: an integral hinge, a fold, a local weakening (e.g. a partial transverse cut), and a joint. Each possibility represents a separate embodiment of the invention. Proximal connection region 26 may be configured for allowing motion of second clip arm 14 relative to first clip arm 12. Proximal connection region 26 may be integrally formed with at least one of clip arms 12 and 14. Optionally, tail coupler 22b is connected to, or is emerging from, first clip arm 12 and may be in proximity with proximal connection region 26. Tail coupler 22b is configured to maintain a constant shape or/and an alignment with first clip arm 12 when second clip arm 14 is moved or/and is positioned apart relative to first clip arm 12.

Hemostatic clip 10 may further include a barrier 32 which emerges distally from or above proximal connection region 26. Optionally, barrier 32 blocks tissue from receiving by or contacting the proximal connection region 26. Barrier 32 may be further configured to provide lateral stability to clip arms 12 and 14. Barrier 32 may be further configured to resist normal sideway moments and/or to provide increased control of the clip during its deployment.

First clip arm 12 is optionally adjoins proximal connection region 26 with a rigid extension 37 characterized by a first cross sectional area 34, which is sized to provide substantial rigidity sufficient to withstand deformation under normal stresses (of clip deployment and manipulation within a subject's body). First cross sectional area 34 may have a length dimension that ranges between about 0.2 mm and about 4 mm.

Second clip arm 14 adjoins proximal connection region 26 with a flexible extension 35 that includes a pliant portion 38. Pliant portion 38 is characterized by a second cross sectional area 36, which is sized to provide substantial pliancy under normal stresses. First cross sectional area 34 may be greater than second cross sectional area 36 by about 1.5 to about 5 times. Pliant portion 38 is optionally more flexible than other portions of the hemostatic clip, and it is optionally elastic, sufficiently to allow autonomous compressing of the clip arms when not subject to negating external stresses. Pliant portion 38 may be displaced and extend from a position adjacent to proximal connection region 26 towards second clip arm distal end 18, or it may be distant from proximal connection region 26 by no more than 0.1, 0.5, 1, 1.5 or 2 millimeters. Pliant portion 38 may have a length that ranges between about 0.2 millimeter and about 3 millimeters. Optionally, pliant portion 38 has a thickness smaller than the minimal thickness of other portions of hemostatic clip 10. The thickness of pliant portion 38 may be within the range of between about 0.1 millimeter and about 2 millimeters. The width of pliant 38 portion may be smaller than the minimal width of other portions of the hemostatic clip. The flexibility of pliant portion 38 may relate to a specific mechanical, chemical or/and thermal treatment applied thereto. Suitable mechanical treatments made to pliant portion 38 may include, but are not limited to abrasion, perforations, cold work (e.g., less than 30%). The flexibility of pliant portion 38 may relate to a specific thermal treatment applied thereto, including, but not limited to a prolonged heat treatment (e.g., at above 420° C.). Optionally, the mechanical and/or thermal treatment may not be applied to other portions of the hemostatic clip.

Flexible extension 35 of second clip arm 14 may include an inclined portion 40 that, relative to a proximal to distal long axis, is curved or bent with a declined angle (towards first clip arm 12) until reaching an inflection point 15 (which is optionally the apex of inclined portion 40) from which it deviates into inclined angle (away first clip arm 12). Inclined portion 40 is located distally to pliant portion 38, optionally sharing boundary region. Inclined portion 40 may be more rigid than pliant portion 38 or have similar rigidity thereto.

Inclined portion 40 provides a relatively large opening of the clip 10, thus allowing a relatively large bodily organ or tissue ligation. Inclined portion 40 may be positioned immediately adjacent to pliant portion 38. Inclined portion 40 may be curved, or bent. Inclined portion 40 may be characterized with stiffness substantially greater than the stiffness of other portions of the hemostatic clip. Inclined portion 40 may have a length within the range of about 0.3 millimeter and about 4 millimeters.

Hemostatic clip 10 further includes an inner resilient elastic clip arm member 20 connected to at least one of first and second clip arms 12 and 14 (shown in both). Inner resilient elastic clip arm 20 is configured to conform to various thicknesses of bodily organs or tissues. By “configured to conform”, it is meant that inner resilient elastic clip arm member 20 is changed in shape in response to ligating a bodily tissue or organ. Inner resilient elastic clip arm member 20 may be bendable and may expand towards a bodily organ or tissue ligated by hemostatic clip 10. Inner resilient elastic clip arm member 20 may be made of an elastic material. Inner resilient elastic clip arm member 20 may be made of a biocompatible material. Optionally, at least one of clip arms 12 and 14 include an inner resilient elastic clip arm member 20. Further optionally, at least one of clip arms 12 and 14 includes an outer rigid clip arm member 24 connected to inner resilient elastic clip arm member 20. Further optionally, each of clip arms 12 and 14 includes an outer rigid clip arm member 24 connected to an inner resilient elastic clip arm member 20. Inner resilient clip arm member 20 may include a tissue contacting surface (not shown). The tissue contacting surface may be made from a material different from the material of inner resilient clip arm member 20. Tissue contacting surface may facilitate bodily organ or tissue grasping.

Reference is now made to FIGS. 2A-2C, which show schematic side views of hemostatic clip 10 according to some embodiments of the invention.

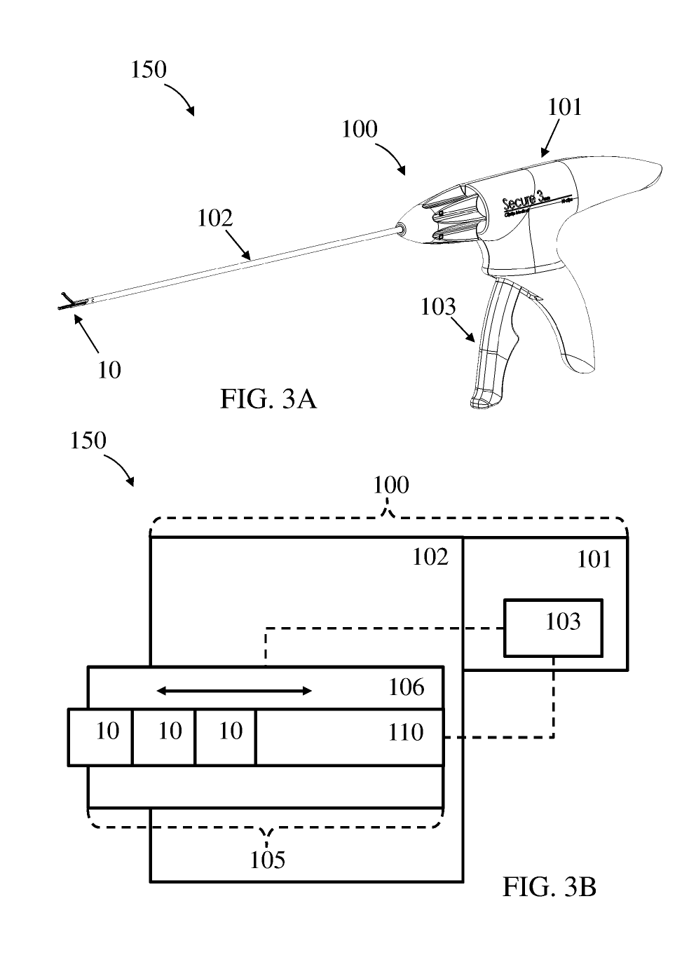

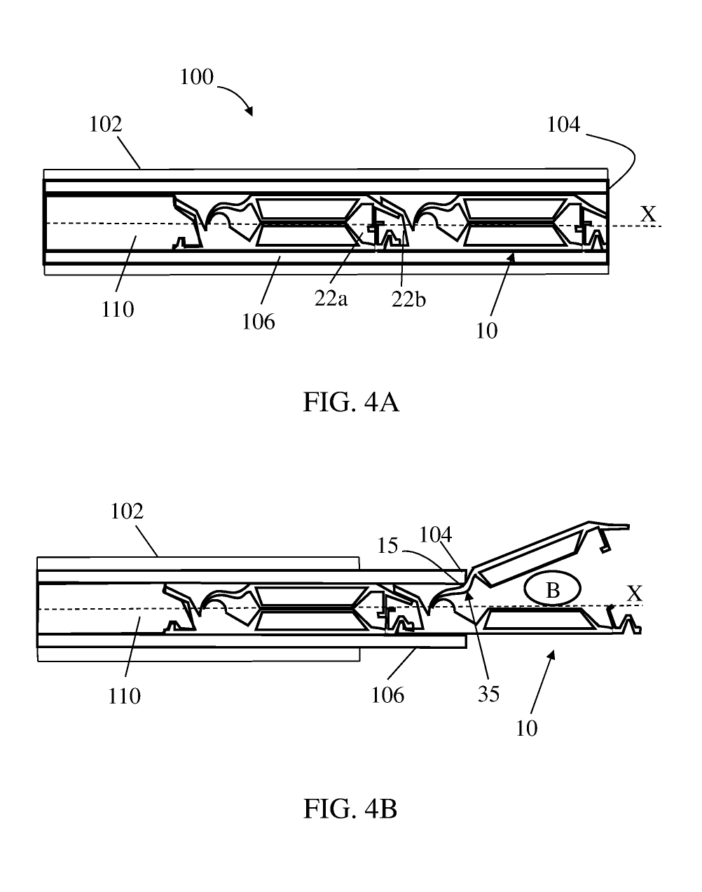

With respect to FIG. 2A, hemostatic clip 10 is shown in a stowed (pre-deployed) configuration which is especially efficient for installing one or more clips in a surgical clip applier (such as clip applier 100 shown in FIG. 3). When the flexible extension 35 of second clip arm 14 is kept pressed against first clip arm 12, across inflection point 15, the first and second clip arms can be compressible into, or be still within boundaries of, the stowed configuration, in which the first and second clip arms are unlockable with each other. When in its stowed configuration, the clip arms can move (closer or farther from each other) within the boundaries of the clip applier, without the risk of unintentional locking. In order to apply the hemostatic clip for ligating a bodily tissue or organ, it is first required to shift hemostatic clip 10 from the stowed configuration to a deployed (e.g., opened) configuration, as shown in FIG. 2B.

Hemostatic clip 10 includes an interconnecting mechanism 22 for connecting to another clip like hemostatic clip 10. Interconnecting mechanism 22 includes a head coupler 22a releasably connectable to a tail coupler 22b, thereby allowing sequential chaining lengthwise a plurality of hemostatic clips 10. Head coupler 22a includes opposing first head coupler arm 22c and second head coupler arm 22d extending generally distally from first and second clip arms 12 and 14, respectively. First head coupler arm 22c includes a deflecting (convex) interconnecting member 22i. Second head coupler arm 22d is in a form of a distal tilting element extending from a distal portion of surgical (hemostatic) clip 10, juxtaposing to second clip arm 14 and tilting towards first clip arm 12.

First head coupler arm 22c and second head coupler arm 22d are configured for engaging mating first and second tail coupler arms 22e and 22f, respectively. First tail coupler arm 22e includes a distal deflecting (concave) interconnecting member 22j configured for mating (e.g., receiving or gripping) with convex interconnecting member 22i. Second tail coupler arm 22f is in a form of a proximally tilting element extending from a proximal portion of surgical (hemostatic) clip 10, juxtaposing to second clip arm 14 and tilting away from first clip arm 12. First and second tail coupler arms 22e and 22f extend generally proximally from each hemostatic clip 10. According to some embodiments, head coupler 22a of a first proximal clip is connected to tail coupler 22b of a subsequent distal clip within snugly fitting stiff boundaries, such that first and second tail coupler arms 22e and 22f press first and second head coupler arms 22c and 22d, respectively.

Second head coupler arm 22d may include an inclined portion (a coupler extension) extending distally with respect to second clip arm 14. Second tail coupler arm 22f may include an inclined portion (a coupler extension) extending proximally with respect to the proximal portion of the distal clip. First head coupler arm 22c and first tail coupler arm 22e are sized and configured for stiff engagement upon head coupler 22a being connected to tail coupler 22b. Coupler extension of second head coupler arm 22d is sized and configured for elastic bending affected by the mating bent extension of inclined extension of second tail coupler arm 22f upon connecting head coupler 22a to tail coupler 22b. Optionally and alternatively, coupler extension of second head coupler arm 22f is sized and configured for elastic bending affected by the mating bent extension of inclined extension of second tail coupler arm 22d upon connecting head coupler 22a to tail coupler 22b. When head coupler 22a is connected to tail coupler 22b, interconnecting mechanism 22 is configured for releasing tail coupler 22b from head coupler 22a by allowing coupler extension of head coupler 22a to elastically deform into a less stressed form while pushing away mating (parallel thereto) inclined extension of tail coupler 22b. Optionally and alternatively, interconnecting mechanism 22 is configured for releasing tail coupler 22b from head coupler 22a by allowing coupler extension of tail coupler 22b to elastically deform into a less stressed form while pushing away mating (parallel thereto) inclined extension of head coupler 22a.

Convex interconnecting member 22i is positioned distally with respect to first head coupler arm 22c. Concave interconnecting member 22j is positioned proximally with respect to first tail coupler arm 22e. Interconnecting mechanism 22 is configured, for releasably locking interconnecting members 22i and 22j when head coupler 22a is connected to tail coupler 22b. According to some embodiments, when interconnected, interconnecting members 22i and 22j allow pivotal motion therebetween. According to some embodiments, interconnecting mechanism 22 is configured, when coupler extensions disengages one from another, for rotating tail coupler 22b relative to head coupler 22a.

Surgical (hemostatic) clip 10 is autonomously shiftable from the stowed configuration to a deployed configuration in which first and second clip arms, 12 and 14, are distant from each other and unlocked. Optionally, when flexible extension 35 of second clip arm 14 is kept pressed against first clip arm 12, from any region thereof proximally to inflection point 15, the first and second clip arms can be compressible into a locked configuration in which the first and second clip arms are locked with each other. FIG. 2B shows hemostatic clip 10 in a deployed configuration, where it is with non-stressed fully opened position of the clip arms. Optionally, in the deployed configuration, first clip arm distal end 16 is distanced from second clip arm distal end 18 by a predetermined distance. The predetermined distance may be the maximal distance or height displayed between first clip arm distal end 16 and second clip arm distal end 18 and may range between about 2 mm and about 30 mm.

Surgical (hemostatic) clip 10 includes a locking mechanism 28, which includes two locking units 28a and 28b provided, each, to first and second clip arms 12 and 14, respectively. Locking units 28a and 28b include mating locking members 28c and 28d, respectively, configured to engage with each other. Locking member 28c faces toward a distal end of the surgical clip 10 and locking member 28d faces towards a proximal end of the surgical clip 10. Each of locking units 28a and 28b further includes a contacting (e.g., seamless or smooth) surface S oppositely facing a respective one of the mating locking members 28c and 28d.

Hemostatic clip 10 and locking mechanism 28 are configured such that, in the stowed configuration (as shown in FIG. 2A, for example), first and second clip arms 12 and 14 are in proximity and locking units 28a and 28b are elastically stressed in a deformation such that contacting surfaces S contact each other and the mating locking members 28c and 28d are non-engaged with each other. In the deployed configuration (as shown in FIG. 2B, for example), first and second clip arms 12 and 14 are distant from each other and locking units 28a and 28b are unstressed and therefore subject to locking, as opposed to their state in the stowed configuration. As such, first and second clip arms 12 and 14 are compressible from the deployed configuration into a locked configuration (as shown in FIG. 2C, for example), in which locking units 28a and 28b are absent of same deformation as in the stowed configuration, and the mating locking members 28c and 28d are engaged with each other.

Locking mechanism 28 of hemostatic clip 10 includes a first locking unit 28a and a second locking unit 28b. First locking unit 28a protrudes inwardly from first clip arm 12 towards second clip arm 14. Second locking unit 28b protrudes inwardly from second clip arm 14 towards first clip arm 12. These locking units 28a and 28b may be positioned any way along the length of the arms 12 and 14, respectively. First locking unit 28a includes first locking member 28c extending distally therefrom. Second locking unit 28b includes second locking member 28d extending proximally therefrom.

In a stowed configuration second locking unit 28b is positioned proximally to first locking unit 28a. In this configuration, first clip arm distal end 16 is in approximation to second clip arm distal end 18 and contacting (e.g., smooth) surfaces S of first and second locking units 28a and 28b press one against the other. Optionally, second locking unit 28b includes an elastic portion. Further optionally, first locking unit 28a imposes shape deformable stresses to the elastic portion of second locking unit 28b when second locking unit 28b is proximal to first locking unit 28a. As used herein the term “shape deformable stresses” refers to reversible changes in the shape due to applied compressive (pushing) forces. Optionally, upon conversion from the stowed configuration (FIG. 2A) to the non-stressed fully open configuration (FIG. 2B), second locking unit 28b shifts distally relative to first locking unit 28a and the shape deformable stresses are eliminated. Locking mechanism 28 members may each be integrally structured and manufactured from a single piece of material of at least one of first clip arm 12 and second clip arm 14. Alternatively, locking mechanism 28 members may be made of materials different from the materials of first clip arm 12 and second clip arm 14.

At least when in the non-stressed fully open configuration, first locking unit 28a is distanced from proximal connection region 26 by a first distance, and second locking unit 28b is distanced from proximal connection region 26 by a second distance. First distance may have a length within the range of between about 0.3 mm and about 40 mm. Second distance may be the same as the first distance or, greater than first distance by about 0.05 millimeter, about 0.1 millimeter, about 0.2 millimeter, about 0.3 millimeter, about 0.4 millimeter, about 0.5 millimeter, about 1 millimeter, about 5 millimeters, about 10 millimeters, about 15 millimeters, about 20 millimeters, about 25 millimeters, about 30 millimeters or about 35 millimeters. Each possibility represents a separate embodiment of the invention.

Upon shifting from the stowed configuration to the deployed (e.g., fully opened) configuration, second clip arm 14 is pivotally movable relative to first clip arm 12. Optionally, a clip-deploying opening angle α extends between second clip arm 14 and first clip arm 12 following movement of second clip arm 14 relative to first clip arm 12. As used herein the term “clip-deploying opening angle α” relates to a maximal angle (i.e., angle α) defined by the maximal opening of the second clip arm 14 relative to the first clip arm 12. According to some embodiments, clip-deploying opening angle α is defined by the maximal opening between inclined portion 40 of second clip arm 14 and a longitudinal axis of first clip arm 12. Optionally, alternatively or additionally, clip-deploying opening angle α is determined directly from an inclined portion angle β formed by inclined portion 40 around inflection point (e.g., apex) 15 thereof. Optionally, clip-deploying opening angle α equals, approximately or exactly, to inclined portion angle β. According to some embodiments, second locking leg 28b is completely elevated relative to first locking leg 28a upon the movement of second clip arm 14 relative to first clip arm 12.

According to some embodiments, second locking leg 28b shifts distally relative to position or/and shape thereof from a pre-deployed configuration to a non-stressed fully open configuration such that at the non-stressed fully open configuration clip-deploying opening angle α extends between inclined portion 40 and first clip arm 12.

With respect to FIG. 2C, hemostatic clip 10 is shown in a stressed locked configuration. In this configuration, hemostatic clip 10 locks together first and second clip arms 12 and 14, respectively, thereby ligating bodily organ or tissue B.

Second clip arm 14 is movable relative to first clip arm 12 from a non-stressed fully opened clip configuration, wherein first clip arm distal end 16 is distanced from second clip arm distal end 18 by a predetermined distance, to a stressed locked clip configuration, wherein first clip arm distal end 16 is in proximity with, or contacts second clip arm distal end 18.

Locking together first and second clip arms 12 and 14, includes interlocking first and second locking members 28a and 28b.

Locking mechanism 28 is configured for applying resistive force to at least one of first clip arm distal end 16 and second clip arm distal end 18, thereby locking together first clip arm distal end 16 and second clip arm distal end 18. Locking mechanism 28 is configured such that when in the closed configuration, although resistive forces are operated on the hemostatic clip, opening of the hemostatic clip is prevented. In the stressed locked clip configuration, first and second clip arms 12 and 14, respectively apply compression and clamping forces to bodily organ or tissue B via at least one of inner resilient elastic clip arm member 20, while allowing at least one of inner resilient elastic clip arm member 20 to expand towards a respective outer rigid clip arm member 24, such that in response to ligating bodily tissue or organ B, first and second clip arms 12, and 14, respectively conform to a compressed and clamped shape of bodily organ or tissue B. In response to ligating a bodily organ or tissue B, or to the elastic deformation of inner resilient elastic clip arm member 20, outer rigid clip arm member 24 prevents deformation of clip arms 12 and 14 and/or limits expansion of inner resilient elastic clip arm member 20.

According to some embodiments, in the stowed configuration or/and in the locked configuration, the mating locking members 22c and 22d are distanced from each other with a first gap G1. According to some embodiment, G1 is defined as the cross sectional distance between first and second head coupler arms 22c and 22d and is being a length that ranges between 0.3 millimeter and 4 millimeters. According some embodiments, in the stowed configuration or/and in the locked configuration, tail coupler arms 22e and 22f are distanced from each other with a second gap G2 being a length that ranges between 0.5 millimeter and 5 millimeters. According to some embodiments, G2 is equal to or greater than G1. According to some embodiments, G2 is greater than G1 by about 0.2 to about 1 millimeter.

FIG. 3A is an illustration shown an isometric view of an exemplary surgical clipping system 150 that includes an exemplary clip applier 100 and at least one (shown as distal-most) surgical clip 10. FIG. 3B is a block diagram representation of surgical clipping system 150, illustrating an exemplary functional arrangement of components in clip applier 100 carrying a plurality of surgical clips 10. 4A-4D are schematic side cut views of a plurality of sequentially chained hemostatic clips carried in clip applier 100, representing exemplary steps in a method for deploying (distal-most) surgical clip 10.

Clip applier 100 is configured for deploying surgical (e.g., hemostatic) clips onto a bodily organ or tissue in various stages of operation. Clip applier 100 is configured to deliver and deploy at least one hemostatic clip 10, or a plurality of hemostatic clips 10, including a distal most hemostatic clip 10. Clip applier 100 may house approximately 20 hemostatic clips, or any number of clips below or above 20 clips. The hemostatic clips are chained lengthwise, one after the other along longitudinal axis X of clip applier 10 (shown in FIG. 4).

Clip applier 100 includes an elongated body 102 which extends along longitudinal axis X (shown in FIG. 4). Elongated body 102 may be in a form of a hollow needle and may include a sharp edge at its distal tip for facilitating selective surgically piercing maneuver through a skin or/and a bodily wall, into a body cavity.

Clip applier 100 further includes a clip-deploying mechanism 105 configured for activating several steps for deploying distal-most surgical clip 10. The clip-deploying mechanism is optionally actuatable manually using a trigger 103 that is pivotally connected to a clip applier console 101. Clip-deploying mechanism 105 includes an introducer 106 configured for sliding and advancing in elongated body 102 along longitudinal axis X. Introducer 106 is capable of sliding and retracting, and may either be stopped at any point in between, or/and be shifted between pre-set positions, relative to elongated body 102 and distal-most surgical clip 10, in accordance with deployment sequence of the surgical clip. FIG. 4A shows introducer 106 in a fully retracted position in which distal-most surgical clip 10 is fully covered and kept in the stowed configuration previously described. FIG. 4B shows introducer 106 in a partially protruding position that partially uncovers distal-most hemostatic clip 10 and allowed to shift autonomously into deployment configuration, as previously described. FIG. 4C shows re-covering of distal-most surgical clip 10 by introducer 106, after it underwent the deployment configuration, thereby shifting surgical clip 10 into locked configuration by forcing clip arms 12 and 14 into contact and locking. FIG. 4D shows a complete uncovering of distal-most surgical clip 10 by introducer 106 allowing it to autonomously release from proximally adjacent surgical clip it was interconnected with, and to thereby eject from clip applier 100.

Clip-deploying mechanism 105 also includes a pusher 110 which is releasably connected to a proximal-most in the interconnected chain of surgical clips 10, and facilitates the axial motion of introducer 106 relative to distal-most surgical clip 10 in all the described positions. In some exemplary embodiments, pusher 110 remains fixed relative to elongated body 102 or console 101, while introducer 106 extends or retracts as described, although in other exemplary embodiments the pusher 110 may be axially slidable with introducer 106 fixed or movable as well.

Referring particularly to a deployment configuration of distal-most surgical clip 10 shown in FIG. 4B, a distal tip 104 of introducer 106 is shown positioned in contact with flexible extension 35 of second clip arm 14 allowing it enough room to pivot away from first clip arm 12 of the distal-most clip 10. In an exemplary embodiment, in order to facilitate affective pivoting of second clip arm 14, for affording the deployment configuration of the clip, the clip applier 100 is configured to position introducer distal tip 104 at the inclined portion 40 of second arm 14, optionally particularly at or adjacent (slightly distal or proximal) to flection point 15. In some exemplary designs or configurations, the exact positioning of distal introducer tip 104 adjacent to inflection point 15 may facilitate a maximal affective opening of the distal-most surgical clip 10, while maintaining overall stability of the clip and the complete surgical clipping system 150, also when affecting a bodily tissue or organ B. In some such embodiments, maximal (allowed/affective) opening of clip 10 forms clip-deploying opening angle α (as shown in FIG. 2B), particularly when distal tip 104 is at (e.g., approximately or exactly) inflection point 15, which is determined directly from (e.g., equals to) inclined portion angle β formed by inclined portion 40 around inflection point 15.

Ligating a bodily tissue or organ includes gradually closing hemostatic clip 10 over bodily organ or tissue B, by forcibly decreasing distance between clip arm 12 and clip arm 14, or between clip arm distal end 16 and clip arm distal end 18.

Clip applier 100 and hemostatic clip 10 contained therewith are configured for selectively and stably align first clip arm 12 to elongated body 102 with longitudinal axis X of hemostatic clip applier 100. Particularly, interconnecting mechanism 22 is configured such that when distal-most hemostatic clip 10 is chained to another hemostatic clip 10 proximally adjacent thereto, the other hemostatic clip(s) 10 affects distal-most hemostatic clip 10 into an alignment with longitudinal axis X. This alignment is maintained throughout bodily tissue or organ B ligation. For example, when hemostatic clip 10 protrudes from distal tip 104, second clip arm 14 deviates from alignment with respect to longitudinal axis X of hemostatic clip applier 100, while first clip arm 12 is maintained aligned with longitudinal axis X of hemostatic clip applier 100. This allows, shifting of hemostatic clip 10 from the stowed configuration to the deployed configuration. Upon ligating bodily organ or tissue B, second clip arm 14 is movable relative to first clip arm 12 from the deployed configuration, wherein first clip arm distal end 16 is distanced from second clip arm distal end 18 by a predetermined distance, to a stressed locked configuration, wherein first clip arm distal end 16 is in proximity with or contacts second clip arm distal end 18.

The particular structure of the current hemostatic clip of the invention in which the first clip arm 12 is maintained stably aligned with the longitudinal axis X of the hemostatic clip 10, affords safe and efficient bodily organ or tissue B ligation. Further, this structure allows affecting other treatments on bodily organ or tissue B, such as dissection thereof. Further, the interconnecting mechanism 22 is configured to overcome the problem of hemostatic clips pressing against the wall of the lumen of the clip applier and thereby increasing friction while laterally sliding within a lumen of hemostatic clip 100. The interconnecting mechanism 22, as structured, reduces normal forces between the clip arms and the inner lumen of the shaft of the clip applier and provides smooth movements with reduced frictions of clips 10 when sliding along the lumen of the hemostatic clip.

Projection or dislodgment of distal most hemostatic clip 10 (shown in FIG. 4D) is mediated following retraction of introducer 106 to the proximal end of elongated body 102. Interconnecting mechanism 22 is configured to unlock mating locking elements 22i and 22j positioned on head coupler 22a and tail coupler 22b, respectively. According to some embodiments, mating locking elements 22i and 22j allow pivotal motion therebetween. According to some embodiments, interconnecting mechanism 22 is configured, for rotating tail coupler 22b relative to head coupler 22a to thereby mediate release of distal most hemostatic clip 10. Elements 22d and 22f, in which one of them is under stressed reversible elastic deformation permit pushing out the distal clip, when the interconnection between the clips is exposed and outer constrain of the housing tube is released. Interconnecting mechanism 22 thus affords manually or automatic (by the change of the interconnecting mechanism, from stressed reversible elastic configuration to non-stressed configuration) controllable release of hemostatic clips by rotating the distal clip about a pivot to thereby release the distal most clip.

According to some embodiments, first clip arm 12 connection to the proximal elements (proximal connection region, hinge portions etc.) of the clip is characterized with stiffness substantially greater than of a soft tissue. Further optionally, hemostatic clip applier 100 is configured for substantially maintaining first clip arm 12 alignment when manipulating soft tissue or body organ B with first clip arm distal end 16.

Clip arms 12 and 14 are configured to apply compression and/or clamping forces towards bodily organ or tissue B via at least one of inner resilient elastic clip arm members 20 in response to ligating a bodily organ or tissue. According to some embodiments, the pressure applied is constant. According to some embodiments, the pressure applied linearly rises in relation to the stage of the bodily organ or tissue ligation. According to some embodiments, the pressure applied depends on the bodily organ or tissue thickness. Optionally, during the course of ligation and prior to becoming restricted in expansion by outer rigid clip arm member 24, at least one of inner resilient elastic clip arm members 20 applies a pressure of up to about 150 gr/mm² on the bodily organ or tissue. Further optionally, when in close contact with outer rigid clip arm member 24, the at least one of inner resilient elastic clip arm members 20 applies a pressure of above 150 gr/mm² on the bodily organ or tissue.

Further optionally, the at least one inner resilient elastic clip arm members 20 applies a compressive pressure of at least 5 gr/mm², at least 10 gr/mm² or at least 15 gr/mm² when a distance of at least 0.1 mm separates between a first and a second inner resilient elastic clip arm members 20, or between a first inner resilient elastic clip arm member 20 and an opposite clip arm member. According to some embodiments, the at least one inner resilient elastic clip arm member 20 applies a compressive pressure in a range of between about 10 gr/mm² to about 150 gr/mm², when a distance in a range of between about 0.2 mm and about 0.8 mm separates between a first and a second inner resilient elastic clip arm member 20, or between a first inner resilient elastic clip arm member 20 and an opposite clip arm member.

FIGS. 4A to 4D provides scenarios representing steps in methods for delivering a surgical clip in a body of a subject, or/and for affecting or ligating a bodily organ or tissue in the subject. A clip applier is provided that may be similar or identical to clip applier 100 described herein. A clip applier includes hemostatic clips, that may be similar or identical to surgical (hemostatic) clip 10 described herein. According to some embodiments, affecting a bodily organ or tissue includes at least one of: dissecting the bodily organ or tissue, and guiding clip applier 100 using first clip arm distal end 14 in relation to the bodily organ or tissue. Introducing the clip applier in the subject's body prior to clips deployment takes place when all surgical clips are in a stowed position, fully covered with introducer 106 within elongated body 102 (as shown in FIG. 4A).

As shown in FIG. 4B, distal-most surgical clip 10 is advanced to protrude at a selected distance from introducer distal tip 104, while aligning first clip arm 12 along longitudinal axis X and allowing second clip arm 14 to pivot away from first clip arm 12, putting the clip in deployment configuration. Clip advancing may include positioning introducer distal tip 104 so as to contact flexible extension 40 adjacent to inflection point 15. When in the deployed configuration (FIG. 4B), the clip applier 100 can be guided in the subject's body using the aligned first clip arm 12, which can also be used (having sufficient stabilized and rigid fixation and alignment with clip applier 100) for dissecting a bodily organ or tissue in the subject's body. In some embodiments, the guiding may include observing distal portion extending from first clip arm 12 in relation to a target bodily organ or tissue in the subject's body.

As also shown in FIG. 4B, distal-most surgical clip 10 is advanced, in its deployed (opened) configuration into surrounding the (target) bodily organ or tissue. Then, as shown in FIG. 4C, it can be forced (using introducer 106) into gradually closing over the bodily organ or tissue by forcibly decreasing distance between first and second clip arms 12 and 14, and to locking together first and second clip arms 12 and 14. In some embodiments, closing and locking the clip 10 includes positioning introducer distal tip 104 distally to inflection point 15.

As shown in FIG. 4D, Distal-most surgical clip 10 is ejected from clip applier 100. As the aligning of first clip arm 12 includes gripping (proximal, convex) deflected interconnecting member 22j, and elastically bending proximal tilting element 22f towards deflected interconnecting member 22j, clip 10 ejecting will optionally include releasing of proximal tilting element 22f from its elastic deformation (bending).

The clip applier may be inserted through a passage (optionally enclosed with a laparoscopic port) for inserting the clip applier into an in-body location. Alternatively, the clip applier may be inserted into an in-body location directly percutaneously

Affecting or ligating a bodily organ or tissue may include surrounding the bodily organ or tissue with distal-most surgical (hemostatic) clip 10, gradually closing distal most hemostatic clip over the bodily organ or tissue by forcibly decreasing distance between first and second clip arm distal ends; and locking together first and second clip arm distal ends 16 and 18. Gradually closing distal most hemostatic clip over the bodily organ or tissue may include shifting introducer 106 from a fully retracted position, proximal to the distal-most hemostatic clip, to a fully protruded position, covering partially or completely the distal-most hemostatic clip arms. Shifting from a fully retracted position, proximal to the distal-most hemostatic clip, to an advanced/protruded position covering and pressing the clip arms, may cause the height between the distal ends of the two clip arms to be reduced and limited to the closed and locked position of the clip. Shifting the introducer to a fully protruded position over the hemostatic clip may initiate activating of the locking mechanism of the hemostatic clip.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of.’

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments. Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A surgical clip comprising:
a first clip arm pivotally connected to a second clip arm at a proximal connection region, said first and second clip arms have locking units including a first locking unit protruding from said first clip arm towards said second clip arm and a second locking unit protruding from said second clip arm towards said first clip arm;
said first locking unit including a first locking member and said second locking unit including a second locking member, and wherein said first locking member is configured to engage with said a second locking member; and said first locking unit further includes a first contacting surface oppositely facing said first locking member and said second locking unit includes a second contacting surface oppositely facing said second locking member;
the surgical clip has a stowed configuration in which said first and second clip arms are in proximity and said locking units are elastically stressed in a deformation such that said first and second contacting surfaces contact each other and said first and second locking members are non-engaged with each other; the surgical clip is shiftable from said stowed configuration to a deployed configuration in which said first and second clip arms are distant from each other, and wherein said first and second clip arms are forcible from said deployed configuration into a locked configuration in which said first and second locking units are absent of said deformation and said first and second locking members are engaged with each other.

2. The surgical clip of claim 1, wherein said first locking member faces toward a distal end of the surgical clip and second locking member faces towards a proximal end of the surgical clip.

3. The surgical clip of claim 1, wherein in said locked configuration said first and second locking members are in a space between said first and second clip arms.

4. The surgical clip of claim 1, wherein said first and second locking units are configured to shift from said deformation to a state absent of said deformation autonomously when said first and second arms are shifted from said stowed configuration to said deployed configuration.

5. The surgical clip of claim 1, wherein said first and second locking members are configured engage each other when said first and second arms are shifted from said deployed configuration to said locked configuration.

6. The surgical clip of claim 1, wherein in said stowed configuration a surface of contact between said first and second contact surface is substantially parallel to a direction of said protruding of said locking units and said first and second locking units are deformed by a stress substantially normal to said surface of contact and in said locked configuration said first and second locking members apply a resistive force preventing opening of the surgical clip to said deployed configuration and wherein said resistive force is substantially parallel to said direction of said protruding.

7. The surgical clip according to claim 1, wherein said contacting surfaces are smooth and configured for sliding with respect to each other.

8. The surgical clip according to claim 1, wherein said surgical clip is forcible from said deployed configuration into said locked configuration by compression force of said first arm towards said second arm.

9. The surgical clip according to claim 8, wherein in said stowed configuration said surgical clip is not forcible into said locked configuration by said compression force of said first arm towards said second arm.

10. The surgical clip according claim 1, further comprising an interconnecting mechanism for connecting to another surgical clip.

11. The surgical clip of claim 1, wherein said first clip arm includes a first outer rigid member and said second clip arm includes a second outer rigid member and said locking members are located between said first outer rigid member and said second outer rigid member.

12. A method for deploying a surgical clip, the method comprising:
providing a clip applier, comprising an introducer lumen extending along a longitudinal axis, and configured for housing at least one surgical clip including a distal-most surgical clip;
retaining said surgical clip inside said lumen in a stowed configuration in which a first clip arm and second clip arm are in proximity and a first locking unit and a second locking unit are elastically stressed in a deformation that prevents said first locking unit from engaging with said second locking unit;
advancing said distal-most surgical clip to protrude at a selected distance from said lumen to allow said distal-most clip to shift autonomously to a deployed configuration in which said first and second clip arms are distant from each other and in which said first and second locking units are absent of said deformation;
closing said arms from said deployed configuration to a locked configuration thereby engaging said first locking unit with said second locking unit; and
applying resistive force from said engaged first and second locking units to at least one of the clip arms, thereby locking together the clip arms during the locked configuration.

13. The method of claim 12, wherein said first locking unit includes a first locking member and said second locking unit includes a second locking member and wherein said first locking member is configured to engage with said second locking member; and wherein said first locking unit further includes a first contacting surface oppositely facing said first locking member and wherein said second locking unit further includes a second contacting surface oppositely facing said second locking member; and wherein in said stowed configuration, contact between said first and second contacting surfaces prevents said engaging of said first and second locking members and wherein shifting of said clip to said deployed configuration further includes separating of said first and second contacting surfaces.

14. The method of claim 12, further comprising an interconnection between said distal-most surgical clip and an interconnected chain surgical chips and wherein said closing results from an axial motion of an introducer with respect to said distal-most clip and wherein said axial motion of said introducer relative to said distal-most clip is facilitated by said interconnection.

15. A surgical clip comprising:
a first clip arm pivotally connected to a second clip arm at a proximal connection region, said first clip arm including a first locking unit having a first locking member and said second clip arm including a second locking unit having a second locking member; said first locking member configured to engage with said second locking member, said first locking unit further including a first contacting surface oppositely facing said first locking member; said second locking unit further including a second contacting surface oppositely facing said second locking member
the surgical clip has a stowed configuration in which said first and second clip arms are in a closed configuration in proximity and said locking units are elastically stressed in a deformation such that said contacting surfaces contact each other and said locking members are non-engaged with each other; the surgical clip is shiftable from said stowed configuration to a deployed configuration in which said first and second clip arms are in an open configuration distant from each other, wherein said first and second clip arms are forcible from said open configuration back into said into said closed configuration while said locking units remain absent of said deformation and said locking members are engaged with each other.

16. The surgical clip of claim 15, wherein said first clip arm includes a first outer rigid member and said second clip arm includes a second outer rigid member and said locking members are located between said first outer rigid member and said second outer rigid member.

17. The surgical clip of claim 15, wherein in said closed configuration said first and second locking members are in a space between said first and second clip arms.

18. The surgical clip of claim 15, wherein said first and second locking units are configured to shift to a state absent of said deformation autonomously when said first and second arms are shifted from said closed configuration to said open configuration.

19. The surgical clip of claim 18, wherein said first and second locking members are configured engage each other autonomously when said first and second arms are shifted from said open configuration to said closed configuration.

20. The surgical clip of claim 15, wherein said first and second locking members face opposite each other.
